(19) United States
(12) Patent Application Publication
(10) Pub. No.: US 2022/0381760 A1
(43) Pub. Date: Dec. 1, 2022

(54) UNMANNED ROBOT FOR WATER QUALITY MANAGEMENT AND METHOD OF CONTROLLING SAME

(71) Applicant: Artwa Co., Ltd, Daedeok-gu (KR)

(72) Inventors: Dong Woo KANG, Jung-gu (KR); Ik Jae SONG, Dong-gu (KR); Jeong Yeon KIM, Yuseong-gu (KR); Hee Jun SHIN, Seo-gu (KR); Dong Hyeon HWANG, Daedeok-gu (KR); Won Gyu SHIM, Seo-gu (KR)

(21) Appl. No.: 17/531,028

(22) Filed: Nov. 19, 2021

(30) Foreign Application Priority Data
May 31, 2021 (KR) 10-2021-0070463

(51) Int. Cl. G01N 33/18; B63B 35/32; G05D 1/02; B62D 55/116; G01S 17/89

(52) U.S. Cl. CPC G01N 33/1886; B63B 35/32; G05D 1/0219; G05D 1/0248; G05D 1/0225; B62D 55/116; G01S 17/89; B63B 2035/007

(57) ABSTRACT

Disclosed is an unmanned robot for water quality management. An unmanned robot for water quality management according to the present disclosure includes a traveling unit provided with a pair of caterpillar treads mounted on both side surfaces of a body and including floating bodies having their own buoyancy, a drive motor that drives the traveling unit, a water quality measurement sensor that measures water quality of a water surface on which the unmanned robot for water quality management is operated, and a processor that controls the traveling unit so that the unmanned robot travels on the water surface to collect water quality data measured by the water quality measurement sensor.

### CROSS-REFERENCE TO RELATED APPLICATION

This application claims priority to and the benefit of Korean Patent Application No. 10-2021-0070463 filed on May 31, 2021, the disclosure of which is incorporated herein by reference in its entirety.

### BACKGROUND

### 1. Field of the Invention

The present disclosure relates to an unmanned robot for water quality management and a method of controlling the same, and more particularly, to an unmanned robot for water quality management, which may be operated in an amphibious manner to remove floating matter on a water surface and collect water quality data, and a method of controlling the same.

### 2. Discussion of Related Art

Water quality and environmental conditions of reservoirs, lakes, ponds, and the like are monitored in various manners to ensure aesthetic aspects and protect ecological resources. In particular, when the management of ponds installed for landscaping in a city park is insufficient, a foul odor occurs due to green algae, and the aesthetic aspects are adversely affected due to floating matter such as various leaves and garbage.

In the related art, a small amount of water is collected to analyze the water quality or a sensor is fixedly installed at a specific location at which the water quality is to be measured and a manager checks the sensor to identify the water quality. However, only a specific point may be measured, and thus it is difficult to measure the water quality, a pollution change amount, or the like of the entire area.

Accordingly, in recent years, a method of measuring the water quality in real time by driving an unmanned robot for measuring the water quality over a water surface has been introduced. In this case, the unmanned robot acquires water quality data while traveling on the water surface in a propeller manner or a water wheel manner. The unmanned robot using the propeller manner or the water wheel manner is not easy to rotate when a direction is changed, and thus mobility thereof is reduced. Due to a high speed and waves of water, the unmanned robot may acquire merely the water quality data in line units in a wide area, but it is difficult for the unmanned robot to acquire accurate water quality data in surface units.

In particular, the unmanned robot using the propeller manner or the water wheel manner requires a certain water depth to be operated on the water surface and changes its direction in a turning manner, and thus the unmanned robot cannot be operated in small reservoirs, ponds, lakes, or the like having a shallow water depth or a small width.

Therefore, the need for an unmanned robot that is easily operated even in small reservoirs, ponds, lakes, and the like and collects the water quality data more effectively is increasing.

### SUMMARY OF THE INVENTION

The present disclosure is directed to providing an unmanned robot for water quality management, which may be operated smoothly even in a small reservoir or pond and may more effectively collect water quality data, and a method of controlling the same.

The technical aspects of the present disclosure are not limited to the aspects described above, and those skilled in the art will clearly understand other technical aspects not described from the following descriptions. One aspect of the present disclosure provides an unmanned robot for water quality management, the unmanned robot including a traveling unit provided with a pair of caterpillar treads mounted on both side surfaces of a body and including floating bodies having their own buoyancy, a drive motor that drives the traveling unit, a water quality measurement sensor that measures water quality of a water surface on which the unmanned robot for water quality management is operated, and a processor that controls the traveling unit so that the unmanned robot travels on the water surface to collect water quality data measured by the water quality measurement sensor.

The unmanned robot for water quality management may further include a recognition sensor that collects surrounding environment data by detecting a shape of a surrounding geographic feature and a distance to the surrounding geographic feature, wherein the processor may determine a traveling pattern on the basis of at least one of the water quality data collected through the water quality measurement sensor and the surrounding environment data collected through the recognition sensor and control the traveling unit so that the unmanned robot autonomously travels on the water surface according to the determined traveling pattern.

The unmanned robot for water quality management may further include a collection unit that collects floating matter on the water surface through an opening formed in a bottom surface of the body.

The recognition sensor may include a light detection and ranging (LiDAR) sensor that performs map mapping on a surrounding environment, a camera sensor that detects a pre-learned floating matter on the water surface, and a plurality of infrared sensors provided in different directions to detect and avoid an obstacle.

The processor controls the traveling unit so that, when the pre-learned floating matter is detected through the camera sensor, the pair of caterpillar treads change directions and travel in a direction in which the detected floating matter is to be collected through the collection unit.

The unmanned robot for water quality management may further include a gyro sensor, and a center-of-gravity unit that includes the drive motor and a battery and is designed to move forward or rearward in a front-rear direction of the unmanned robot, wherein the processor may control, on the basis of an inclination detected by the gyro sensor, the center-of-gravity unit to move forward or rearward in the front-rear direction to maintain a balance.

The processor may control, when a remaining amount of a battery falls below a preset threshold, the unmanned robot to return to a docking station on land through the recognition sensor, performs docking for charging, moves a floating matter removal plate held on a rear end of the collection unit in a docked state forward, and discharges the floating matter collected through the collection unit.

The unmanned robot for water quality management may further include a communication unit that transmits the collected water quality data to a remote monitoring device in real-time.

The unmanned robot for water quality management may further include a hologram fan disposed at an upper end of the unmanned robot, wherein the processor may control the hologram fan to rotate so as to output a three-dimensional hologram display corresponding to pre-stored input data.

Another aspect of the present disclosure provides a method of controlling an unmanned robot for water quality management, the method including controlling the unmanned robot for water quality management to travel on a water surface by driving a pair of caterpillar treads including floating bodies having their own buoyancy, collecting water quality data obtained by measuring water quality of the water surface through a water quality measurement sensor, and transmitting the collected water quality data to a remote monitoring device in real-time.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a schematic block diagram illustrating a configuration of an unmanned robot for water quality management according to an embodiment of the present disclosure;

FIG. 2 is a perspective view illustrating the overall shape of an internal structure of the unmanned robot for water quality management according to an embodiment of the present disclosure;

FIG. 3 is a view for describing a water quality monitoring system using the unmanned robot for water quality management according to an embodiment of the present disclosure;

FIG. 4 is a side view for describing a floating body located in a traveling unit of the unmanned robot for water quality management according to an embodiment of the present disclosure;

FIG. 5 is a front view for describing a collection unit of the unmanned robot for water quality management according to an embodiment of the present disclosure;

FIGS. 6A and 6B are views for describing a floating matter discharging operation of the collection unit according to an embodiment of the present disclosure;

FIGS. 7A and 7B are views for describing an operation of a center-of-gravity unit of the unmanned robot for water quality management according to an embodiment of the present disclosure;

FIGS. 8A and 8B are views illustrating various external shapes of the unmanned robot for water quality management according to an embodiment of the present disclosure;

FIG. 9 is a view for describing an operation of a hologram fan of the unmanned robot for water quality management according to an embodiment of the present disclosure;

FIGS. 10 and 11 are views for describing a charging station of the unmanned robot for water quality management and a docking method thereof according to an embodiment of the present disclosure; and

FIG. 12 is a flowchart for describing a method of controlling the unmanned robot for water quality management according to an embodiment of the present disclosure.

### DETAILED DESCRIPTION OF EXEMPLARY EMBODIMENTS

First, general terms are selected as terms used in the present specification and the appended claims in consideration of functions in various embodiments of the present disclosure. However, these terms may change according to the intention of those skilled in the art, the legal or technical interpretation, the emergence of new technologies, or the like. Further, some terms may be terms selected by an applicant in a predetermined manner. These terms may be interpreted as the meaning defined in the present specification and may be interpreted on the basis of general content of the present specification and common technical knowledge in the art when there is no specific definition of the terms.

Further, the same reference numerals or symbols in drawings attached to the present specification indicate parts or components performing substantially the same functions. For convenience of description and understanding, even in different embodiments, the description will be made using the same reference numerals or symbols. That is, even when all the components having the same reference numeral are illustrated in a plurality of drawings, the plurality of drawings do not denote one embodiment.

Further, in the present specification and the appended claims, terms including ordinal numbers such as “first” and “second” may be used to distinguish between components. These ordinal numbers are used to distinguish the same or similar components from each other, and the meaning of the terms should not be limitedly interpreted due to the use of the ordinal numbers. As an example, the components combined with these ordinal numbers should not be interpreted as limiting the order of use or arrangement by the numbers. As needed, the ordinal numbers may be used interchangeably.

In the present specification, singular expressions include plural expressions unless clearly otherwise indicated in the context. It should be understood in the present application that terms such as “include” or “configure” are intended to indicate that there are features, numbers, steps, operations, components, parts, or combinations thereof that are described in the specification and do not exclude, in advance, the possibility of the presence or addition of one or more other features, numbers, steps, operations, components, parts, or combinations thereof.

Further, expressions referring to directions such as front/rear/left/right/up/down described in the present specification are defined as illustrated in the drawings, but this is intended to describe the present disclosure to the extent that the present disclosure may be clearly understood, and it is obvious that the directions may be differently defined depending on where a standard is set.

Further, in an embodiment of the present disclosure, when it is described that a first component is connected to a second component, this includes not only direction connection but also indirect connection through a third medium. Further, the fact that a first component includes a second component means that other components are not excluded but may be further included, unless otherwise stated.

Hereinafter, the present disclosure will be described in more detail with reference to the accompanying drawings.

A brief description of each drawing is provided to fully understand the drawings cited in detailed description of the present disclosure.

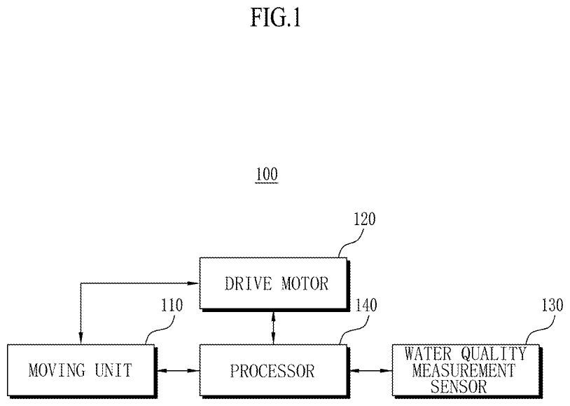

FIG. 1 is a schematic block diagram illustrating a configuration of an unmanned robot for water quality management according to an embodiment of the present disclosure.

The unmanned robot 100 according to the embodiment of the present disclosure is to provide a water quality management service in a prescribed place such as a reservoir, a pond, and a lake, and preferably to function as an autonomous unmanned robot for managing water quality and removing floating matter in small reservoirs or ponds in city parks.

Referring to FIG. 1, the unmanned robot 100 according to the present disclosure includes a traveling unit 110, a drive motor 120, a water quality measurement sensor 130, and a processor 140.

The traveling unit 110 allows the unmanned robot 100 to travel on a land and a water surface in an amphibious manner. In detail, the traveling unit 110 is provided with, on left and right sides of the unmanned robot 100, a pair of caterpillar treads 111 and a plurality of idlers 112 on which the caterpillar treads 111 are wound and thus may perform forward movement, rearward movement, and direction change in a caterpillar tread manner.

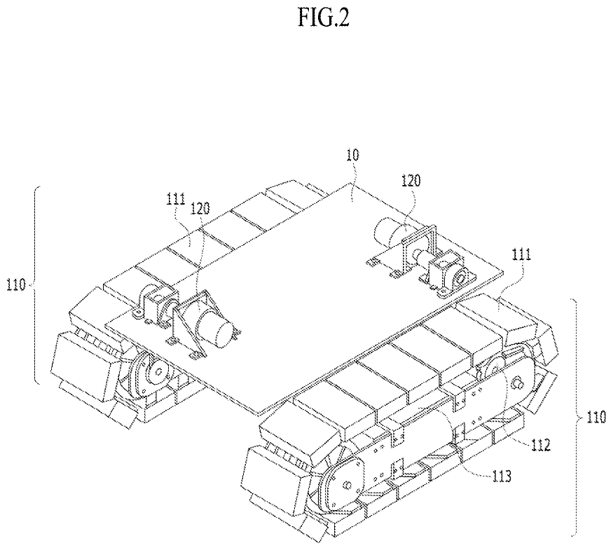

As illustrated in FIG. 2, the caterpillar tread 111 is a device that connects a plurality of plates made of a steel plate in a chain shape, hangs the connected plates on the front and rear idlers 112 like a belt, and rotates and travels by power. As compared to normal wheels, the contact area is large, friction with the ground is large, and thus the unmanned robot 100 may freely travel on rough roads or mud. Further, by changing rotation speeds of the left and right caterpillar treads 111, the direction may be freely changed, and a rotation radius may be reduced to a minimum. That is, when both caterpillar treads 111 rotate in directions opposite to each other at the same speed, the direction may be changed without moving the center of gravity.

In this case, a floating body 113 having buoyancy is provided between the idlers 112 surrounded by the caterpillar treads 111, the traveling unit 110 itself serves as a floating body, and thus the unmanned robot 100 may travel with sufficient buoyancy on the water surface.

The drive motor 120 is configured to convert electric energy into mechanical energy of rotational movement and transmit power so that the traveling unit 110 rotates. The drive motor 120 may be implemented as a direct current (DC) motor, receive electric power from a rechargeable battery (not illustrated) that stores charging electric power, and transmit the power to the traveling unit 110 using a timing belt. A reduction ratio and a torque increase due to the transmitted power, a sprocket connected to the timing belt rotates, the caterpillar treads 111 rotates, and thus the unmanned robot 100 may travel.

Here, a charging terminal of the battery is connected to a commercial power (power outlet or the like in the home) or is electrically connected to the commercial power through a charging part of a docking station 200 being in contact with the charging terminal in a state of being docked in the docking station 200 connected to the commercial power, and thus the battery may be charged.

Electrical components constituting the unmanned robot 100 may receive the electric power from the battery, and thus the unmanned robot 100 may autonomously travel in a state in which the battery is charged.

As illustrated in FIG. 2, a pair of drive motors 120 for driving the left and right traveling units 110 may be provided on a control panel 10 provided on an upper surface of a body to control the unmanned robot 100.

The water quality measurement sensor 130 is configured to measure a state of the water quality of the water surface on which the unmanned robot 100 travels and may acquire water quality parameters such as total dissolved solids (TDS) and pH. The water quality measurement sensor 130 may be attached to a lower part of the unmanned robot 100 partially submerged below the water surface and may acquire various water quality parameters for measuring the water quality.

The water quality measurement sensor 130 may be implemented to acquire various water quality parameters such as dissolved oxygen (DO), oxidation reduction potential (ORP), hydrodeoxygenation (HDO), conductivity, temperature, salinity, turbidity, water depth, chlorophyll a, cyanobacteria, rhodamine, photosynthetic active radiation (PAR), ions, chromophoric dissolved organic matter (CDOM), and crude oil in addition to the above-described water quality parameters.

The processor 140 is configured to control the overall operation of the unmanned robot 100.

In detail, the processor 140 may be provided on the control panel 10 and control the traveling unit 110 so that the unmanned robot 100 travels on the water surface to collect water quality data measured from the water quality measurement sensor 130. In this case, the processor 140 controls the caterpillar treads 111 to rotate in forward/rearward directions or rotate the left and right caterpillar treads 111 in different direction to rotate the unmanned robot 100 in place so as to change a traveling direction.

In this case, the processor 140 may be set so that the unmanned robot 100 travels on the water surface according to a previously input traveling method or travels on the water surface on the basis of a signal detected through a recognition sensor 160.

In detail, the processor 140 may control the traveling unit 110 on the basis of the water quality data collected through the water quality measurement sensor 130 so that the unmanned robot 100 intensively travels in a water quality degradation area in which a water pollution degree exceeds a preset reference value. For example, the processor 140 may determine a traveling pattern in which a traveling speed is reduced so that the unmanned robot 100 stays in the water quality degradation area, in which many pollutants such as green algae are distributed or in which the unmanned robot 100 travels while repeatedly turning around the water quality degradation area, longer and may control the traveling unit 110 so that the unmanned robot 100 autonomously travels on the water surface according to the determined traveling pattern.

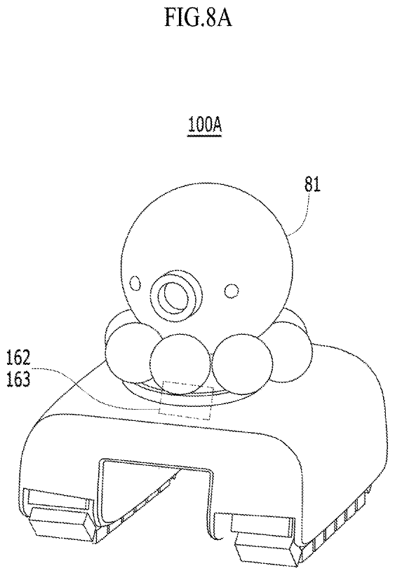

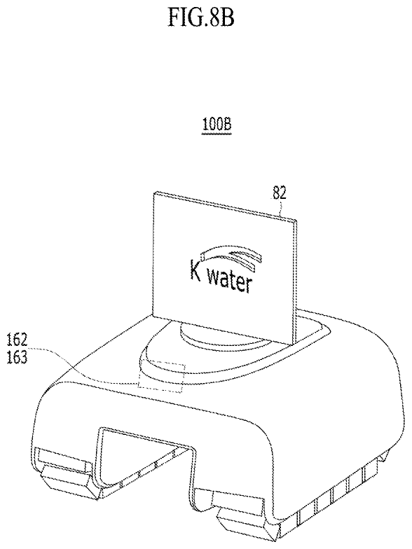

Further, the processor 140 may determine the traveling pattern on the basis of surrounding environment data collected through the recognition sensor 160 and may control the traveling unit 110 so that the unmanned robot 100 autonomously travels on the water surface according to the determined traveling pattern. For example, the processor 140 may detect, through the recognition sensor 160, an area in which foreign substances such as fallen leaves, garbage, and green algae are distributed and may control the traveling unit 110 so that the unmanned robot 100 travels in the corresponding area. A detailed description thereof will be described with reference to FIGS. 8A and 8B.

Further, the processor 140 may further include a communication unit 150 which transmits the collected water quality data to a remote monitoring device 300 in real-time.

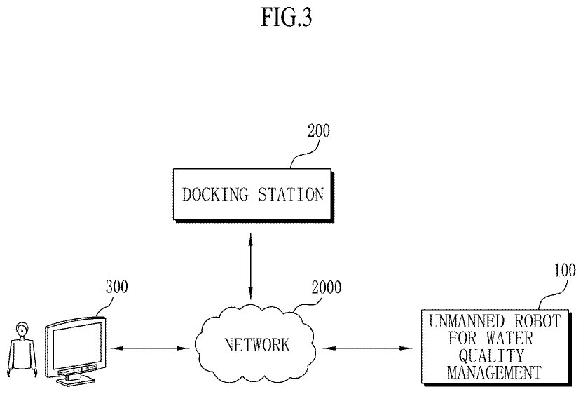

Referring to FIG. 3, a water quality monitoring system 1000 includes the unmanned robot 100, the docking station 200, and the remote monitoring device 300.

The communication unit 150 may communicate with the remote monitoring device 300 through a network 2000 and include a communication module therefor. Here, the network 2000 may be implemented to perform wireless communication using a wireless communication technology such as long-term evolution (LTE), fifth generation (5G), institute of electrical and electronics engineers (IEEE) 802.11 wireless local area network (WLAN), IEEE 802.15 wireless personal area network (WPAN), ultra wide band (UWB), Wi-Fi, ZigBee, Z-wave, and Bluetooth.

A user of the remote monitoring device 300 may identify information on the unmanned robot 100 in the monitoring system 1000 and information on the water quality data collected therefrom through the remote monitoring device 300 or a user terminal such as a personal computer (PC) and a mobile terminal connected to the remote monitoring device 300.

Further, the remote monitoring device 300 may function as a cloud sever or be linked thereto to remotely provide various solutions and content for controlling the unmanned robot 100.

The remote monitoring device 300 may store and manage the water quality data received from the unmanned robot 100. The unmanned robot 100 may acquire the water quality data expressed in gradients in surface units while traveling at a low speed and may transmit the water quality data to the remote monitoring device 300 through the communication unit 150 in real-time. In this case, the remote monitoring device 300 may be a device provided by a manufacturer of the unmanned robot 100 or a company entrusted with a service by the manufacturer.

Further, the remote monitoring device 300 may remotely monitor or control a state of the unmanned robot 100, and a plurality of unmanned robots 100 may be operated to provide a more effective service.

Since a detailed configuration of the communication unit 150 may be easily designed by those skilled in the art to which the present disclosure pertains in order to perform communication through the network 2000, a detailed description thereof will be omitted.

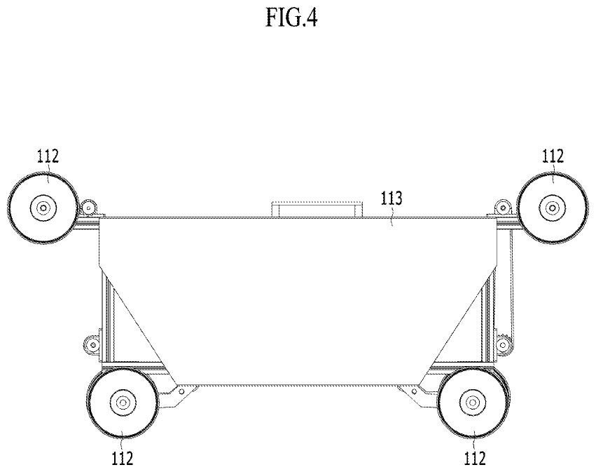

FIG. 4 is a side view for describing a floating body located in a traveling unit of the unmanned robot for water quality management according to an embodiment of the present disclosure.

Referring to FIG. 4, a floating body 114 having its own buoyancy may be included in a space in which the caterpillar treads 111 of the traveling unit 110 are located. The floating body 114 may be made of a material having its own buoyancy, such as Styrofoam. Further, according to an embodiment, the floating body 114 may have a structure in which an internal space is empty or may be implemented as an air tank in which air is injected into a closed internal space.

In this case, since the floating body 114 should have a structure that easily climbs on the land due to the characteristics of the caterpillar treads 111 operated in an amphibious manner, as illustrated in FIG. 4, the floating body 114 preferably has a three-dimensional structure in which an upper surface thereof is relatively longer than a lower surface thereof.

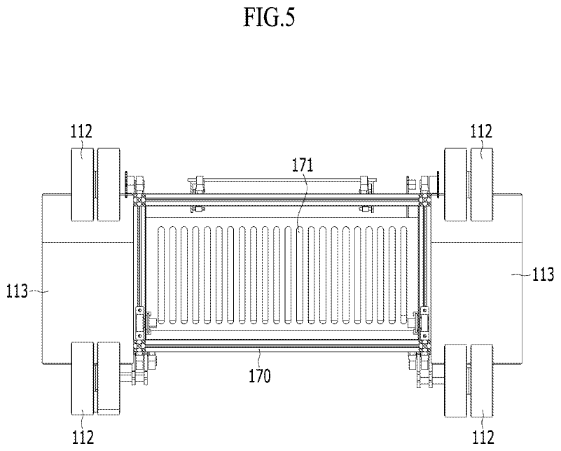

FIG. 5 is a front view for describing a collection unit of the unmanned robot for water quality management according to an embodiment of the present disclosure.

As illustrated in FIG. 5, a collection unit 170 that collects floating matter on the water surface through an opening formed on a bottom surface of the body may be provided. In detail, the collection unit 170 may include an empty space in which the floating matter is collected between the pair of caterpillar treads 111 in the traveling unit 110, a floating matter removal plate 171 for discharging the floating matter to the outside, and a drive motor (not illustrated) that drives the floating matter removal plate 171 forward and rearward.

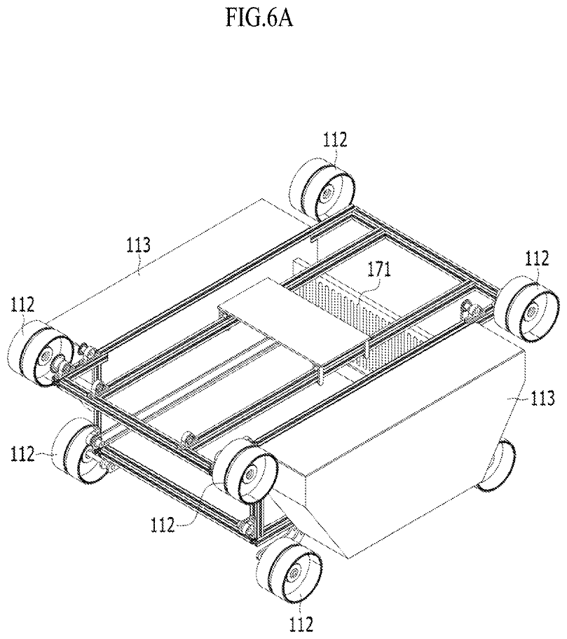

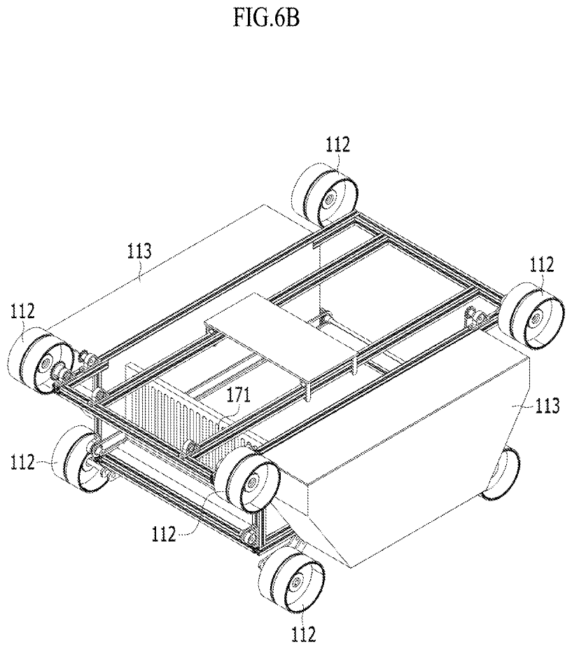

As illustrated in FIG. 6A, the floating matter removal plate 171 is normally situated in the rearmost location, and as the unmanned robot 100 travels on the water surface, floating matter, such as fallen leaves, on the water surface is collected into an empty space between the opening and the floating matter removal plate 171 through the opening. In this case, according to an embodiment, the collection unit 170 may be provided with a support or a net for supporting the collected floating matter at a lower end thereof.

In this case, when the remaining amount of the battery falls below a preset threshold, the processor 140 may control the unmanned robot 100 to return to the docking station 200 on the land to be charged, and as illustrated in FIG. 6B, may control, through the drive motor, the floating matter removal plate 171 located on the rear side in a docked state to move forward, and thus the collected floating matter is discharged to the outside again.

As compared to a propeller method in which a traveling speed is high and many waves of water are generated, in the unmanned robot 100 using a caterpillar tread method according to the present disclosure, since inertia and resistance in the water can be calculated much more easily, the location can be accurately calculated, and thus the floating matter may be more effectively collected.

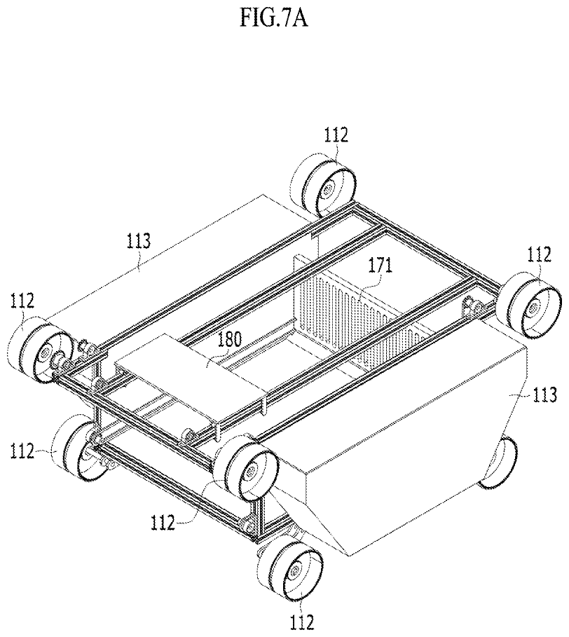

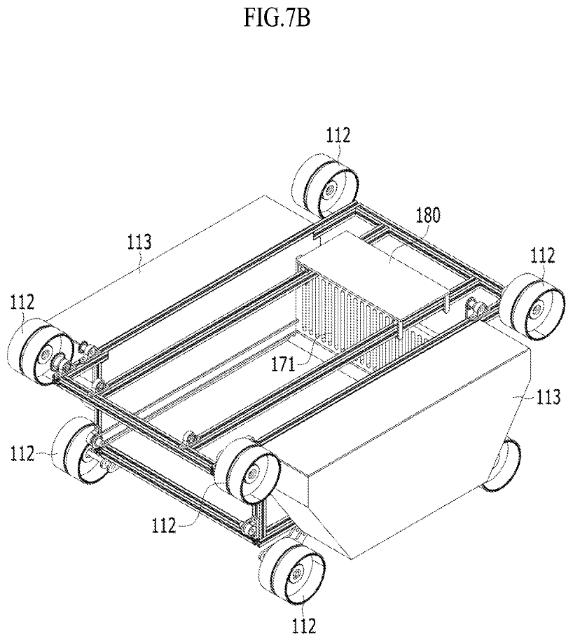

FIGS. 7A and 7B are views for describing an operation of a center-of-gravity unit of the unmanned robot for water quality management according to an embodiment of the present disclosure.

According to an embodiment, the unmanned robot 100 of the present disclosure may further include a center-of-gravity unit 180 and a gyro sensor (not illustrated).

The center-of-gravity unit 180 is disposed in an upper part of the body, performs forward and rearward movements in a front-rear direction, and thus corrects a front-rear inclination of the unmanned robot 100 to maintain balance.

In detail, the center-of-gravity unit 180 includes the drive motor and the battery and thus may secure a certain weight or more for achieving the balance. A casing forming a space in which the drive motor 120 and the battery are accommodated may be provided at an upper portion of the center-of-gravity unit 180.

The processor 140 may control the center-of-gravity unit 180 to move forward or rearward in the front-rear direction on the basis of an inclination detected by the gyro sensor. For example, the processor 140 may control the center-of- gravity unit 180 to be located in a middle of the floating body 113 during a normal time, control the center-of-gravity 180 to move forward as illustrated in FIG. 7A when the unmanned robot 100 is inclined rearward, and control the center-of-gravity unit 180 to move rearward as illustrated in FIG. 7B when the unmanned robot 100 is inclined forward. In this case, a movement distance of the center-of-gravity unit 180 may be proportional to the inclination detected by the gyro sensor.

Accordingly, even when the water surface on which the unmanned robot 100 travels is shaking or the unmanned robot 100 moves on land having an uneven ground surface, the unmanned robot 100 may travel in the balance without overturning.

FIGS. 8A and 8B are views illustrating various external shapes of the unmanned robot for water quality management according to an embodiment of the present disclosure.

An external shape of the unmanned robot 100 may have a structure in which various resistances are reduced in a streamlined shape extending from the front side to the rear side while forming curves on both sides.

However, according to an embodiment, as illustrated in FIG. 8A, an unmanned robot 100A for water quality management in which various sculptures 81 such as figures, character models, and light emitting diode (LED) decorations are attached to the upper part of the body and which thus has an external shape causing familiarity may be provided.

Further, as illustrated in FIG. 8B, an unmanned robot 100B for water quality management in which a lighting panel 82 on which a catch phrase is displayed is attached to the upper part of the body and which may thus transmit a message may be provided.

In this case, all the sculptures 81 and the lighting panel 82 may be implemented in a state of being easily attached or detached through a magnet or the like.

Further, in both the embodiments of FIGS. 8A and 8B, the recognition sensor 160 including a light detection and ranging (LiDAR) sensor 161, a camera sensor 162, and a plurality of infrared sensors 163 may be provided in an upper part and an inside of a support to which the sculptures 81 and the lighting panel 82 are attached.

The recognition sensor 160 is configured to collect the surrounding environment data by detecting the shape and distance of a surrounding geographic feature, and the processor 140 may control the traveling unit 110 on the basis of the surrounding environment data collected through the recognition sensor 160 so that the unmanned robot 100 autonomously travels on the water surface.

In detail, the recognition sensor 160 may include the LiDAR sensor 161 for autonomous driving, the camera sensor 162 for detecting pre-learned floating matter on the water surface, and the plurality of infrared sensors 163 provided in different directions to detect and avoid obstacles.

The LiDAR sensor 161 is a sensor that performs map mapping for a surrounding environment, and when a pulse laser signal radiated from the LiDAR sensor 161 collides with a surrounding object and returns to the LiDAR sensor 161, the surrounding object may be recognized by analyzing the returned pulse laser signal. As a result, a real-time three-dimensional map of the surrounding environment may be generated.

The LiDAR sensor 161 may be disposed at an upper end of the body such as an upper portion of the support to which the sculptures 81 and the lighting panel 82 are attached.

Further, according to an embodiment, the unmanned robot 100 may further include a location acquisition unit (not illustrated) for acquiring current location information. The unmanned robot 100 may include a global positioning system (GPS) module or an UWB module to determine a current location. Accordingly, a more precise map mapping may be performed by additionally using a navigation map obtained through a GPS or the like.

The processor 140 may acquire surrounding map data including space- related data and object-related data collected through the LiDAR sensor 161 and the GPS and transmit the acquired surrounding map data to the remote monitoring device 300.

The camera sensor 162 for detecting the pre-learned floating matter on the water surface may be inserted into a lower end of the LiDAR sensor 161.

The camera sensor 162 is configured to detect the floating matter in front thereof, such as fallen leaves and garbage, on the water surface, and the processor 140 may control the traveling unit 110 so that, when the flowing matter is detected through the camera sensor 162, the unmanned robot 100 travels toward the detected floating matter, and the pair of caterpillar treads 111 change directions and travel in a direction in which the floating matter is to be collected through the collection unit 170.

The processor may learn, in advance, an image of fallen leaves by artificial intelligence in order to detect the floating matter such as fallen leaves or garbage on the water surface.

To this end, the unmanned robot 100 may include an artificial neural networks (ANN) in the form of software or hardware that have been trained to recognize at least one of properties of objects such as users, voices, attributes of spaces, and obstacles.

According to an embodiment of the present disclosure, the unmanned robot 100 may include a deep neural network (DNN) such as a convolutional neural network (CNN), a recurrent neural network (RNN), and a deep belief network (DBN) that are trained by deep learning. For example, the processor 140 or a separate storage unit (not illustrated) of the unmanned robot 100 may be equipped with a DNN structure such as the CNN.

The remote monitoring device 300 may be trained using the DNN on the basis of data received from the unmanned robot 100 and data input by the user and then transmit the updated DNN structure data to the unmanned robot 100. Accordingly, the artificial intelligence DNN structure provided by the unmanned robot 100 may be updated.

Accordingly, when the camera sensor 162 detects fallen leaves on the water surface using an artificial neural network obtained by continuously learning images of the fallen leaves, the processor 140 may control the traveling of the traveling unit 110 so that the collection unit 170 collects the fallen leaves.

Further, according to an embodiment, the unmanned robot 100 may determine a current location using the image acquired through the camera sensor 162.

The plurality of infrared sensors 163 are configured to detect and avoid an obstacle through infrared light, may be inserted into a periphery of the camera sensor 162, and may be inserted into a lower end of the camera sensor 162 in different directions. For example, three to eight infrared sensors 163 may be arranged in different directions of the body.

In this case, the infrared sensors 163 may detect the obstacle according to any one of a light quantity measurement method in which a distance to the obstacle is measured by measuring the amount of infrared light reflected by the obstacle and returning to the infrared sensors 163, a time measurement method in which a time during which the infrared light is reflected by the obstacle and returns to the infrared sensors 163 is measured, and a triangulation method in which a location is tracked by a distance at which the infrared light, which is shot horizontally to the front side, hits the obstacle, and is reflected from the obstacle, is focused on a mirror sensor of the unmanned robot 100.

Accordingly, the approach of animals such as ducks or other unexpected situations may be prevented.

Further, according to an embodiment, the infrared sensors 163 may be used for the unmanned robot 100 to return to the docking station 200 on the ground. When the docking station 200 emits infrared lights having a plurality of different specific wavelengths that cause the unmanned robot 100 to return to the docking station 200, the unmanned robot 100 moves to the docking station 200 using the infrared light emitted from the docking station 200 as a guide.

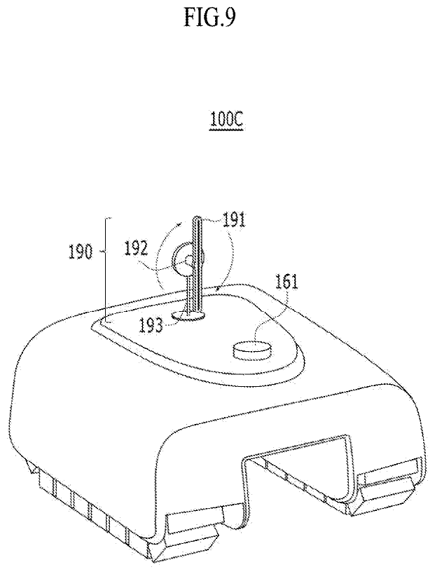

FIG. 9 is a view for describing an operation of a hologram fan of the unmanned robot for water quality management according to an embodiment of the present disclosure.

As illustrated in FIG. 9, a hologram fan 190 is a rotating fan in which light emitting bodies are densely embedded, and as the hologram fan 190 rotates, a three- dimensional holographic shape may be expressed as if the three-dimensional holographic shape is floating in the air.

The hologram fan 190 may include a fan 191, a drive shaft 192, and a support body 193. The hologram fan 190 is disposed above the body, and the processor 140 may control the fan 191 to rotate about the drive shaft 192 so as to output a three-dimensional hologram display image corresponding to pre-stored input data through the light emitting bodies provided in the fan 191. In this case, the light emitting bodies may be implemented as an LED element.

The drive shaft 192 extending from a central portion of the fan 191 may be connected to the support body 193 to form a single body. The hologram fan 190 may further include a fixing member or pressing unit that vertically supports the support body 193 in order to prevent vibrations due to a rotational movement of the fan 191.

The processor 140 may control driving of the hologram fan 190 and the lighting of the light emitting bodies to implement a hologram image. The hologram image expressed through the hologram fan 190 may be expressed in various ways such as advertisements, city catch phrases, various characters, and pictures, and an input unit (not illustrated) and a storage unit (not illustrated) that receive and store data for such a hologram image may be provided.

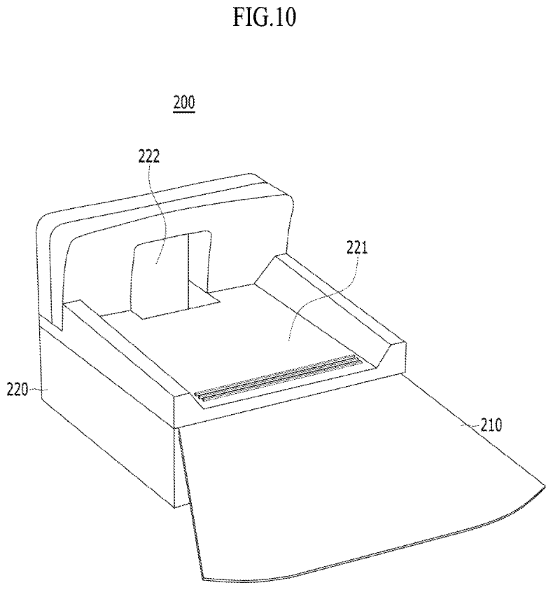

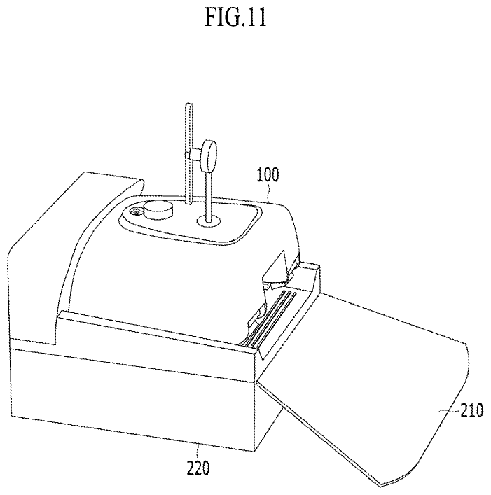

FIGS. 10 and 11 are views for describing a charging station of the unmanned robot for water quality management and a docking method thereof according to an embodiment of the present disclosure.

The unmanned robot 100 may be provided with a charging connection terminal (not illustrated) to be charged with a current from the docking station 200, and according to an embodiment, an inductive or resonant wireless charging pad may be provided.

Further, a bumper (not illustrated) that may absorb an impact when the unmanned robot 100 is docked with the docking station 200 may be additionally installed on a front surface of the unmanned robot 100.

The docking station 200 is a device provided so that the unmanned robot 100 may be docked therewith, and when the unmanned robot 100 is completely docked with the docking station 200, the floating matter collected in the collection unit 170 of the unmanned robot 100 may be automatically discharged.

In detail, when the docking is detected, the processor 140 moves the floating matter removal plate 171 held on a rear end of the collection unit 170 forward, and thus the floating matter collected through the collection unit 170 may be pushed out by the floating matter removal plate 171 and discharged to a discharge container (not illustrated) provided in the docking station 200.

Further, the docking station 200 may function to charge the unmanned robot 100 by supplying a current to the unmanned robot 100 through the charging terminal or the wireless charging pad.

Looking at the configuration, the docking station 200 includes a platform 210 and a housing 220 formed at an end of the platform 210.

The platform 210 is a floor on which the unmanned robot 100 moves and is provided to be inclined so that the unmanned robot 100 may easily climb up and down.

A docking induction device (not illustrated), a processor (not illustrated) and the like may be provided inside the housing 220, and an opening 222 through which the floating matter in the collection unit 170 is discharged to the discharge container and a support part 221 on which the unmanned robot 100 is seated in a docked state may be provided outside the housing 220.

In this case, the wireless charging pad for wireless charging of the unmanned robot 100 may be provided at an upper portion of the support part 221. Accordingly, when the unmanned robot 100 is seated on the support part 221, the wireless charging may be automatically performed. Since a configuration of the wireless charging and operating principles thereof may be sufficiently reproduced by those skilled in the art, a detailed description thereof will be omitted.

Further, the opening 222 of the docking station 200 is provided at a location at which the opening 222 may communicate with the opening in the collection unit 170 of the unmanned robot 100. Accordingly, in the docked state, the floating matter discharged from the collection unit 170 of the unmanned robot 100 may be introduced into the opening 222, and the floating matter introduced into the opening 222 may be introduced into the discharge container of the docking station 200.

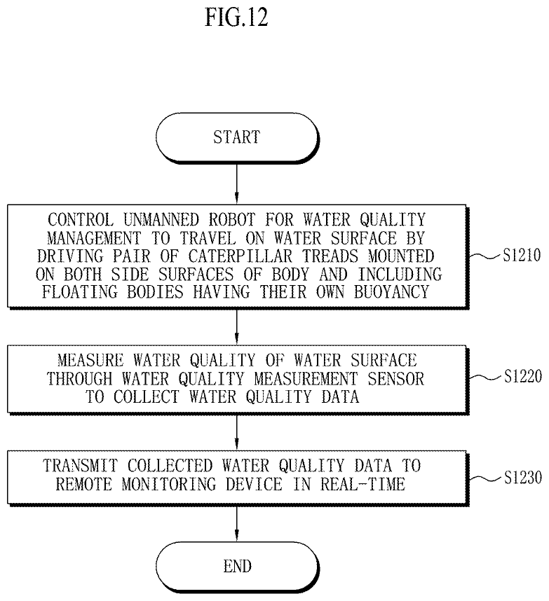

FIG. 12 is a flowchart for describing a method of controlling the unmanned robot for water quality management according to an embodiment of the present disclosure.

First, by driving a pair of caterpillar treads including floating bodies having their own buoyancy, an unmanned robot for water quality management is controlled to travel on the water surface (S1210). In this case, the surrounding environment data is collected by detecting the shape and distance of the surrounding geographic feature through the recognition sensor, and autonomous driving may be performed on the basis of the collected surrounding environment data.

In detail, the recognition sensor may include the LiDAR sensor for performing map mapping on the surrounding environment, the camera sensor for detecting the pre-learned floating matter on the water surface, and the plurality of infrared sensors provided in different directions to detect and avoid the obstacle.

Further, when the floating matter is detected through the camera sensor, the pair of caterpillar treads may be controlled to change the direction thereof and travel in a direction in which the detected floating matter is to be collected through the collection unit.

Further, the center-of-gravity unit may be controlled to move forward or rearward in a front-rear direction to maintain the balance on the basis of the inclination detected by the gyro sensor.

Thereafter, the water quality data is collected by measuring the water quality of the water surface through the water quality measurement sensor provided at a lower end of the unmanned robot 100 (S1220).

Thereafter, the collected water quality data is transmitted to the remote monitoring device in real-time (S1230).

Meanwhile, when the remaining amount of the battery falls below the preset threshold, the unmanned robot may be controlled to return to the docking station on the land through the recognition sensor, to perform the docking for charging, to move the floating matter removal plate held on the rear end of the collection unit in the docked state forward, and to discharge the floating matter collected through the collection unit.

According to the above-described embodiment, the unmanned robot for water quality management may more easily change the direction thereof, may have increased mobility, may become more useful even in small shallow reservoirs and ponds, may collect the water quality data in surface units, and thus may increase the efficiency of data collection.

Meanwhile, various embodiments described in the present specification may be implemented using hardware, software, and/or a combination thereof. For example, various embodiments may be implemented as one or more application specific integrated semiconductors (ASICs), digital signal processors (DSPs), digital signal processing devices (DSPDs), programmable logic devices (PLDs), field programmable gate arrays (FPGAs), processors, controllers, microcontrollers, microprocessors, and other electronic units designed to perform the functions presented herein, or combinations thereof.

Further, for example, various embodiments may be included in or encoded on a computer-readable medium including instructions. The instructions included in or encoded on the computer-readable medium may cause a programmable processor or other processors to perform, for example, an investment information evaluation method according to the present disclosure when the instructions are executed. The computer-readable medium includes a computer storage medium. The storage medium may be a predetermined available medium that may be accessed by a computer. For example, the computer-readable medium may include a random-access memory (RAM), a read-only memory (ROM), an electrically erasable and programmable read only memory (EEPROM), a compact disc read-only memory (CD-ROM) or other optical disk storage mediums, a magnetic disk storage medium or other magnetic storage devices, or a predetermined other medium that may be used to store a desired program code in the form of instructions or data structures accessible by the computer.

The hardware, the software, and the like may be implemented in the same device or individual devices to support various operations and functions described in the present specification. Additionally, in the present disclosure, elements, units, modules, components, and the like described as “parts” may be implemented together or individually as individual but interoperable logical devices. Descriptions of different features of modules, units, and the like are intended to emphasize different functional embodiments and do not necessarily mean that the description should be implemented by individual hardware or software components. Rather, functions related to one or more modules or units may be performed by the individual hardware or software components or may be integrated within common or individual hardware or software components.

Although operations are illustrated in the drawings in a specific order, it should not be understood that these operations need to be performed in a specific order or sequential order illustrated to achieve a desired result or all illustrated operations need to be performed. In a predetermined environment, multitasking and parallel processing may be advantageous. In addition, in the above-described embodiment, it should not be understood that the division of various components is required in all the embodiments, and it should be understood that the described components may generally be integrated together into a single software product or packaged into a plurality of software products.

According to the present disclosure, a direction change of an unmanned robot for water quality management becomes easier, mobility thereof increases, and thus usability increases even in small shallow reservoirs or ponds. Unlike the existing propeller structure in which water quality data is collected in line units, the water quality data can be collected in surface units using a caterpillar tread structure, and thus the efficiency of data collection can increase.

As described above, optimal embodiments have been disclosed in the drawings and the specification. Although specific terms are used herein, the terms are used only for describing the present disclosure and are not used to limit the meaning or the scope of the present disclosure described in the appended claims. Therefore, it should be understood by those skilled in the art that various modifications and other equivalent other embodiments are possible therefrom. Thus, the true technical scope of the present disclosure should be determined by the technical spirit the appended claims.

What is claimed is:

1. An unmanned robot for water quality management, the unmanned robot comprising:
a traveling unit provided with a pair of caterpillar treads mounted on both side surfaces of a body and including floating bodies having their own buoyancy;
a drive motor that drives the traveling unit;
a water quality measurement sensor that measures water quality of a water surface on which the unmanned robot for water quality management is operated; and
a processor that controls the traveling unit so that the unmanned robot travels on the water surface to collect water quality data measured by the water quality measurement sensor.

2. The unmanned robot of claim 1, further comprising a communication unit that transmits the collected water quality data to a remote monitoring device in real-time.

3. The unmanned robot of claim 1, further comprising a recognition sensor that collects surrounding environment data by detecting a shape of a surrounding geographic feature and a distance to the surrounding geographic feature,
wherein the processor determines a traveling pattern on the basis of at least one of the water quality data collected through the water quality measurement sensor and the surrounding environment data collected through the recognition sensor, and controls the traveling unit so that the unmanned robot autonomously travels on the water surface according to the determined traveling pattern.

4. The unmanned robot of claim 3, further comprising a collection unit that collects floating matter on the water surface through an opening formed in a bottom surface of the body. 5

5. The unmanned robot of claim 4, wherein the recognition sensor includes:
a light detection and ranging (LiDAR) sensor that performs map mapping on a surrounding environment;
a camera sensor that detects a pre-learned floating matter on the water surface; and
a plurality of infrared sensors provided in different directions to detect and avoid an obstacle.

6. The unmanned robot of claim 5, wherein the processor controls the traveling unit so that, when the pre-learned floating matter is detected through the camera sensor, the pair of caterpillar treads change directions and travel in a direction in which the detected floating matter is to be collected through the collection unit. 20

7. The unmanned robot of claim 1, further comprising:
a gyro sensor; and
a center-of-gravity unit that includes the drive motor and a battery and is designed to move forward or rearward in a front-rear direction of the unmanned robot,
wherein the processor controls, on the basis of an inclination detected by the gyro sensor, the center-of-gravity unit to move forward or rearward in the front-rear direction to maintain a balance.

8. The unmanned robot of claim 4, wherein the processor controls, when a remaining amount of a battery falls below a preset threshold, the unmanned robot to return to a docking station on land through the recognition sensor, performs docking for charging, moves a floating matter removal plate held on a rear end of the collection unit in a docked state forward, and discharges the floating matter collected through the collection unit.

9. The unmanned robot of claim 1, further comprising a hologram fan disposed at an upper end of the unmanned robot,
wherein the processor controls the hologram fan to rotate so as to output a three-dimensional hologram display corresponding to pre-stored input data .

10. A method of controlling an unmanned robot for water quality management, the method comprising:
controlling the unmanned robot for water quality management to travel on a water surface by driving a pair of caterpillar treads mounted on both side surfaces of a body and including floating bodies having their own buoyancy;
collecting water quality data obtained by measuring water quality of the water surface through a water quality measurement sensor; and
transmitting the collected water quality data to a remote monitoring device in real-time
